(19) United States
(12) Patent Application Publication
(10) Pub. No.: US 2019/0142327 A1
(43) Pub. Date: May 16, 2019

(54) METHOD AND APPARATUS FOR MEASURING SACCADIC LATENCY

(71) Applicant: REBIScan, Inc., Boston, MA (US)

(72) Inventors: Lee Goldstein, Newton, MA (US); Justin Shaka, Boston, MA (US); Robert Winsor, Hamilton, VA (US); Howard Ying, Cambridge, MA (US)

(21) Appl. No.: 16/225,796

(22) Filed: Dec. 19, 2018

Related U.S. Application Data
(63) Continuation of application No. 15/472,462, filed on Mar. 29, 2017.
Application No. 15/472,462 is a continuation of application No. PCT/US17/13116, filed on Jan. 12, 2017.
(60) Provisional application No. 62/278,196, filed on Jan. 13, 2016.

(51) Int. Cl. A61B 5/00; A61B 3/00; A61B 3/113

(52) U.S. Cl. CPC A61B 5/4064; A61B 3/113; A61B 3/0008; A61B 3/0091

(57) ABSTRACT

A device and method for measuring saccadic latency, including a light source positioned within a housing that projects a beam of light through a window of the housing and onto a retina of a subject, lights disposed on or in an external surface of the housing, detectors positioned within the housing and configured to detect light reflected from the retina of the subject through the window of the housing, and a controller that causes output of a sequence of cues and determines a time interval corresponding to a delay between output of a cue in the sequence of cues and either fixation or loss of fixation by the subject, the fixation or loss of fixation being determined based at least in part on the light reflected from the retina of the subject.

### CROSS-REFERENCE TO RELATED APPLICATIONS

The present application is a continuation of U.S. Non-Provisional Application No. 15/472,462, filed Mar. 29, 2017, which itself is a continuation of International Patent Application No. PCT/US17/13116, filed Jan. 12, 2017, which claims priority to U.S. Provisional Patent Application No. 62/278,196, filed Jan. 13, 2016, the disclosures of which are hereby incorporated by reference in their entirety.

### SUMMARY

The present disclosure relates generally to a method and an apparatus for quantitative detection and/or analytical measurement of saccadic eye movements for use in providing a noninvasive biomarker to identify and track brain function or dysfunction and/or predict chronic sequelae following head injuries, such as traumatic brain injury (“TBI”). The present disclosure incorporates by reference, in their entireties, U.S. Pat. Nos. 6,027,216 and 7,959,292.

Brain dysfunction, including injury related to TBI from concussive and subconcussive head trauma, can be difficult to diagnose, as history of such an event is often incomplete and symptoms are nonspecific and overlap with a broad range of neuropsychiatric disorders. Although many patients with dysfunction make a full recovery, a significant subset does not. Individuals that experience multiple mild traumatic brain injuries (“mTBIs”) are at increased risk of persistent post-injury symptoms and long-term complications, including serious sequelae, such as chronic traumatic encephalopathy (“CTE”). Simple interventions, such as removing the patient from risky environments, may prevent these complications by allowing time for the brain to heal and preventing further injury. However, intervention requires prompt and accurate identification of patients at risk. The prior art provides no validated biomarker of brain dysfunction or traumatic brain injury that enables rapid, non-invasive, objective evaluation of this important clinical assessment.

Saccadic velocity can be assessed using eye movement recordings to track the exact position of the eye as the subject (e.g., individual) carries out various tasks (e.g., watching a moving object). Prior art methods involve imaging the anterior structures of the eye or eyes, or placing a contact lens with metallic on the eye or eyes. Prior art methods require intense image processing or a large homogenous magnetic field to determine the accuracy of fixation. Prior art devices are not able to detect precise foveal fixation due to the inability to assess retinal position. The device and method of the present disclosure overcome the above and other disadvantages of the prior art, and accomplish the above and other objectives.

The present disclosure is directed generally to medical devices and/or a method of neurological screening, and more particularly to a retinal scanning system and/or method that measures fixation of each eye individually and misalignment between the two eyes (i.e., microstrabismus). The scanning method of the present disclosure can be extended to measure fixation speed and accuracy and maintenance of binocular alignment to identify brain dysfunction. The present disclosure can also predict increased risk of long-term sequelae of injury, and is complemented by modifications to enable precise measurement of saccadic eye movement. At least the following types of saccades are targeted and/or detectable by the method(s) of the present disclosure: visually guided saccade and anti-saccade.

Retinal birefringence scanning of one embodiment of the present disclosure affords an opportunity to detect with high precision the moment an eye fixates on a target without the need for imaging. By including a saccadic task to a scanner, it is possible through the system and/or method of the present disclosure to determine exactly when the eye leaves the target, and when it returns to the target, without need to determine the location of the eye in the interval between departure and return. This makes it possible to create a considerably simpler saccadic velocity assessment that, when combined with the ability to determine fixation stability and binocular alignment, allows for a rapid and effective probe of ocular dysfunction in patients who have suffered brain injury. To achieve the above, the present disclosure includes: (i) altering the interpupillary distance to optimize for children, young adults, and mature adults; (ii) modifying the optical prescription of the device for eyes of patients of all ages (e.g., children and adults); (iii) lengthening the scanning interval to assess fixation stability; and/or (iv) adding a saccadic task to assess saccadic velocity.

### BRIEF DESCRIPTION OF THE DRAWINGS

The foregoing summary, as well as the following detailed description, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 is a front perspective view of a device in accordance with one embodiment of the present disclosure;

FIG. 2 is a magnified front elevation view of a portion of the device shown in FIG. 1;

FIG. 3 is a perspective view of at least a few internal components of the device of FIG. 1;

FIG. 4 is a top plan view of at least a few internal components of the device of FIG. 1, wherein directional arrows are included to show the flow of light into, through and/or out of the device;

FIG. 5 is a magnified perspective view of a least a few internal components of the device of FIG. 1;

FIG. 6 is another magnified perspective view of a least a few internal components of the device of FIG. 1;

FIG. 7 is a magnified elevation view of a least a few internal components of the device of FIG. 1;

FIG. 8 is a diagram, flow chart and/or algorithm for bionocular fixation detection (convergence) in accordance with one embodiment of the present disclosure;

FIG. 9 is a diagram, flow chart and/or algorithm for monocular fixation detection/fixation stability in accordance with one embodiment of the present disclosure, wherein the steps of FIG. 7 can replace the left-most column (i.e., “calculations”) of FIG. 6 in at least one embodiment of the present disclosure;

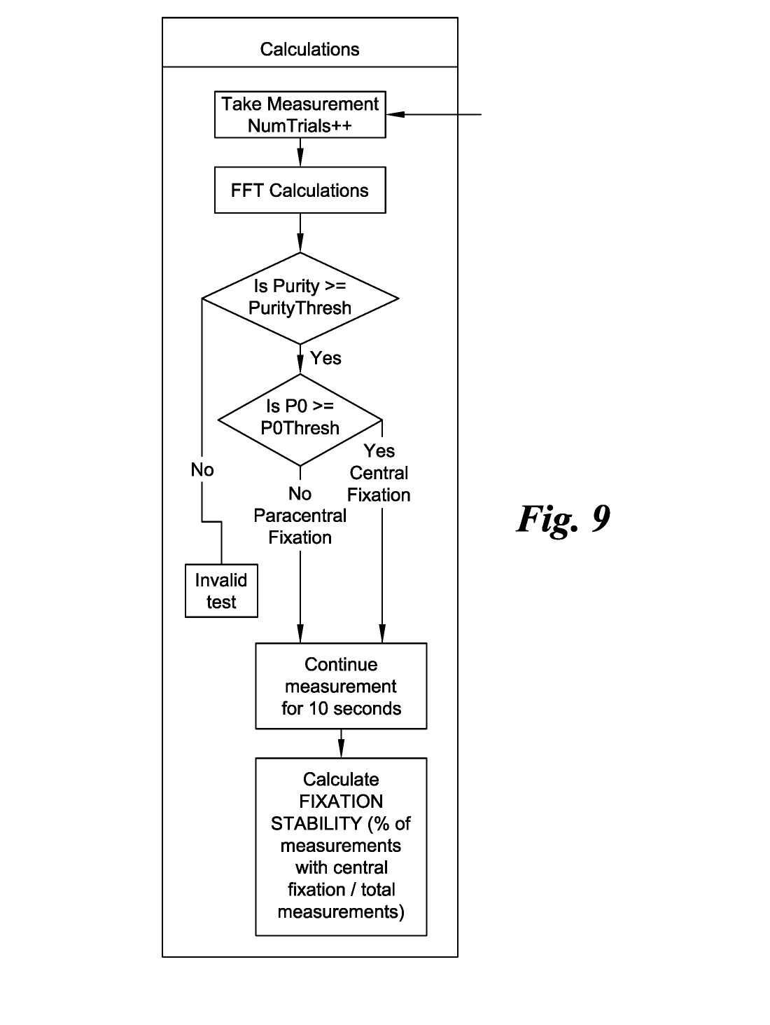

FIG. 10 is a diagram, flow chart and/or algorithm for calculating saccadic latency in accordance with one embodiment of the present disclosure;

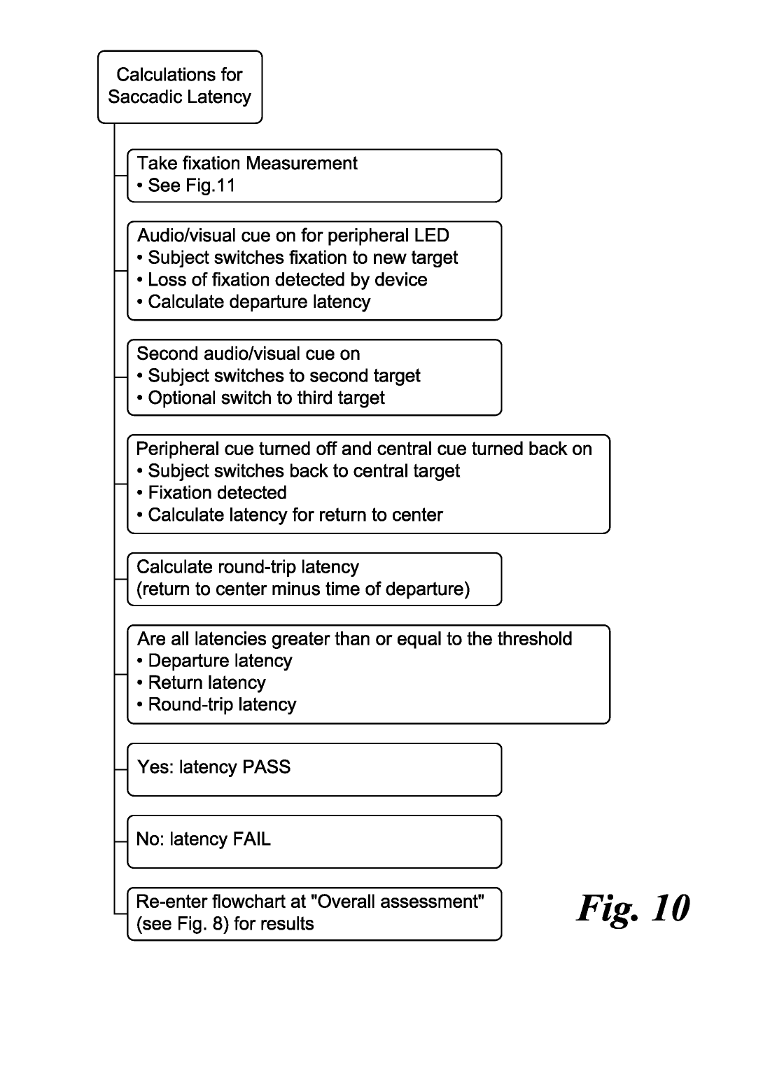

FIG. 11 is a diagram, flow chart and/or algorithm in accordance with one embodiment of the present disclosure; and

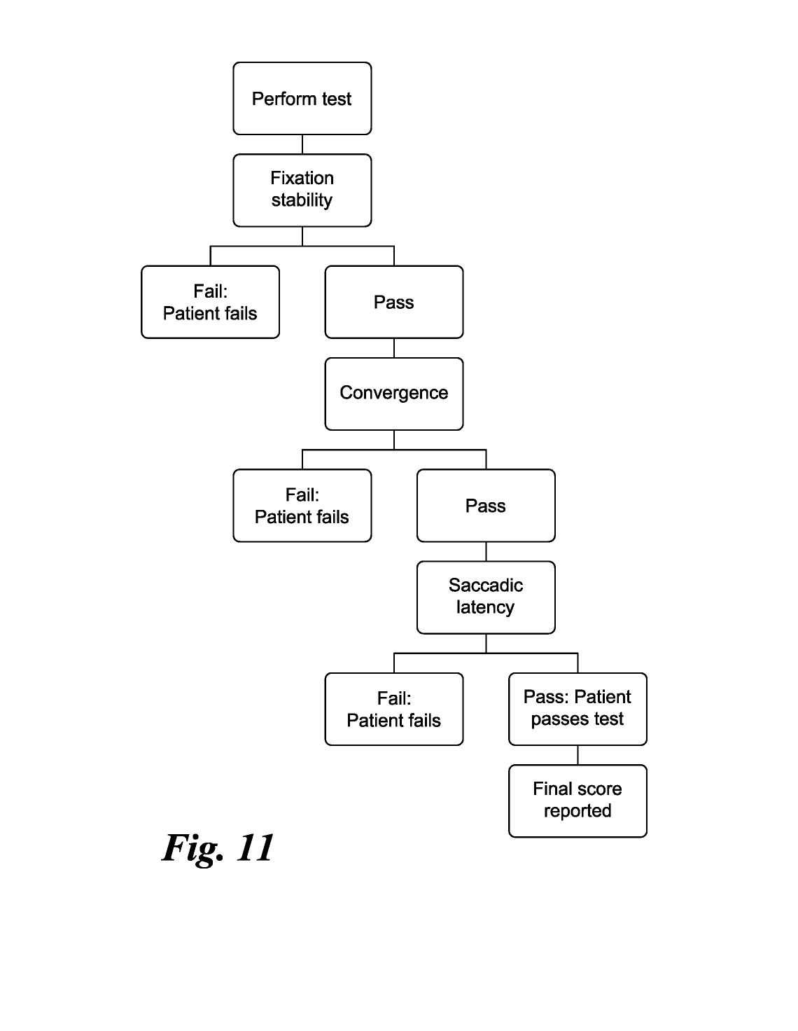

FIG. 12 shows an exemplary computing device useful for performing or initiating processes disclosed herein.

### DETAILED DISCLOSURE

Certain terminology is used in the following description for convenience only and is not limiting. Certain words used herein designate directions in the drawings to which reference is made. Unless specifically set forth herein, the terms “a,” “an” and “the” are not limited to one element, but instead should be read as meaning “at least one.” The terminology includes the words noted above, derivatives thereof and words of similar import.

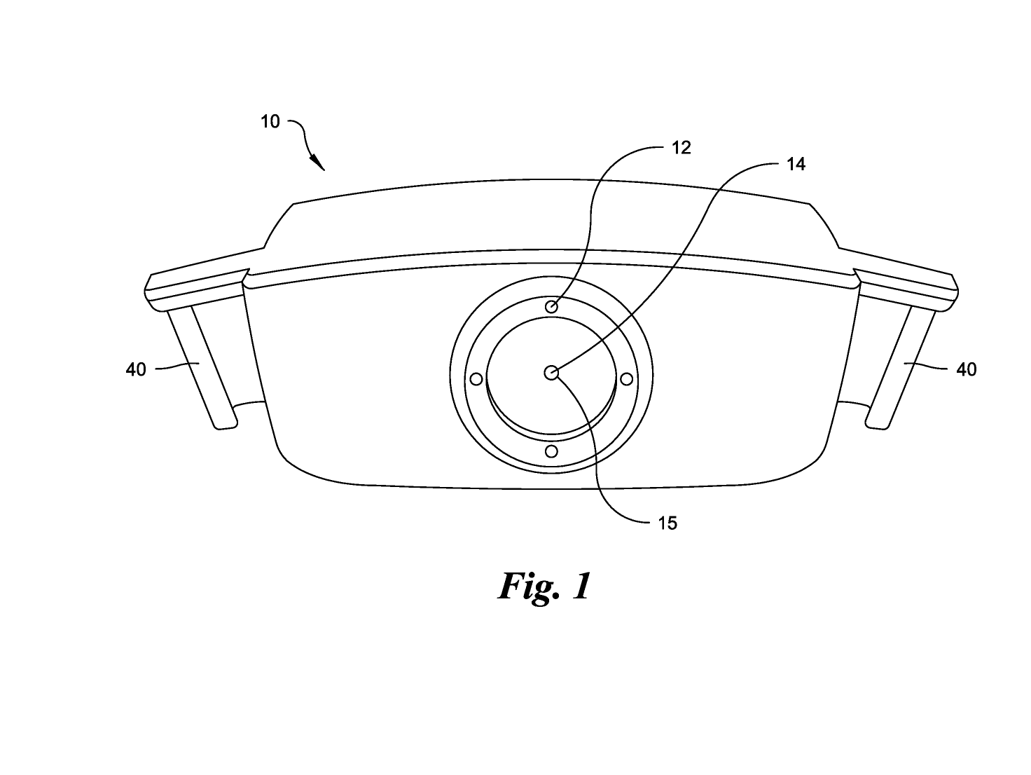

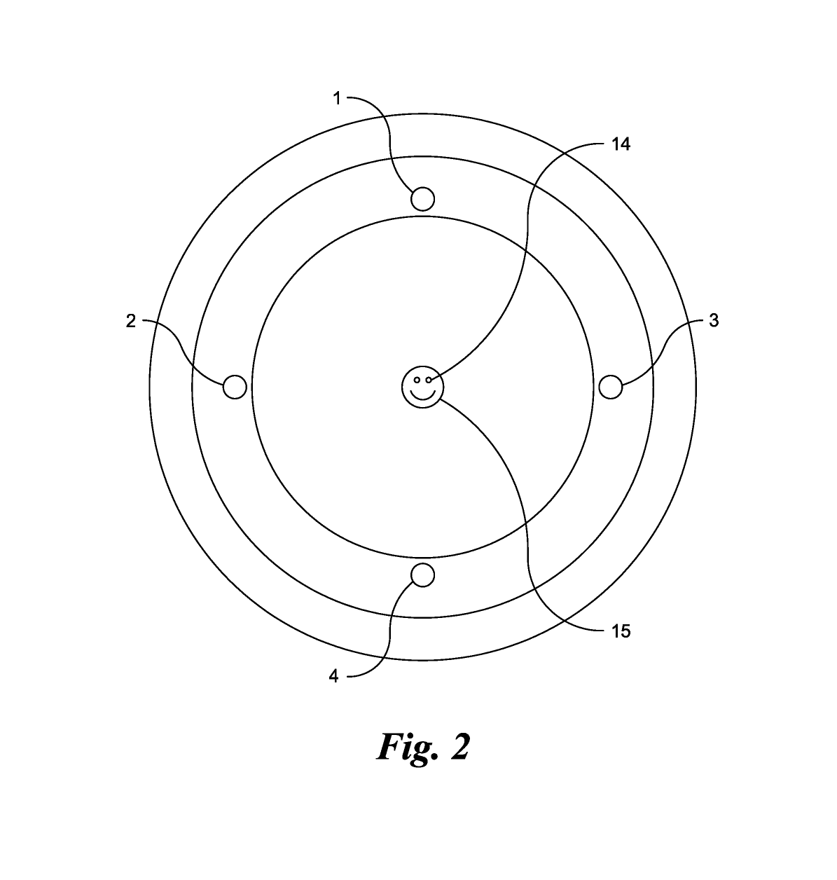

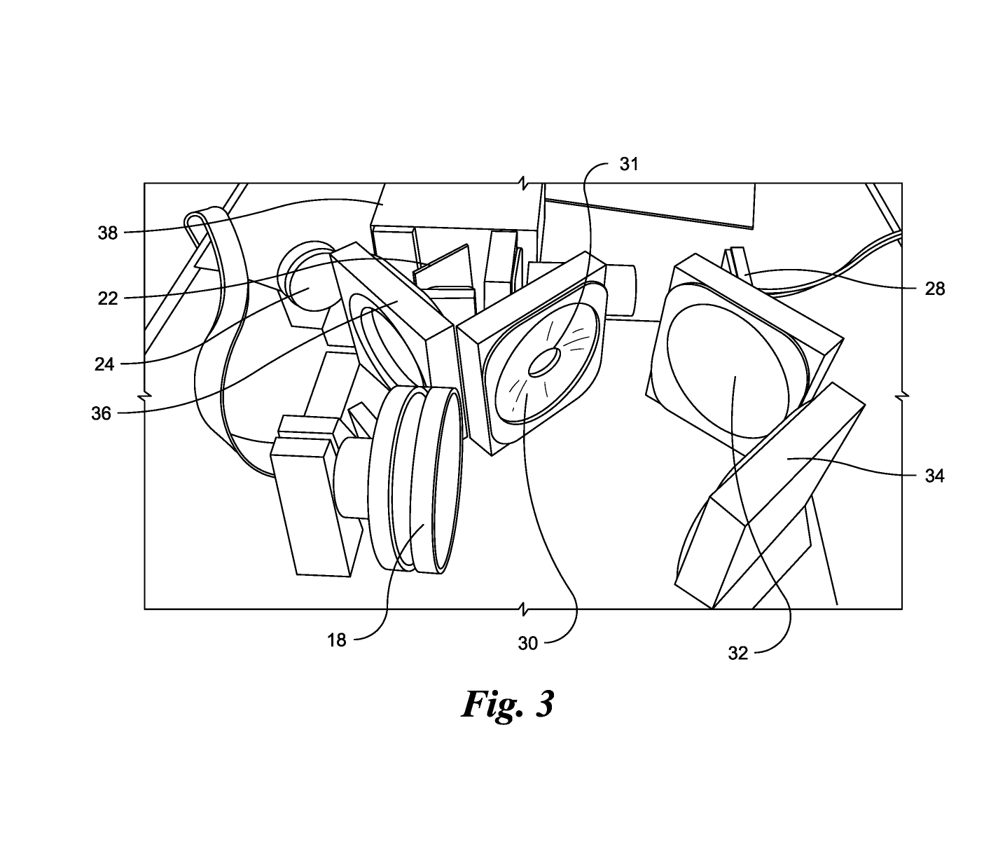

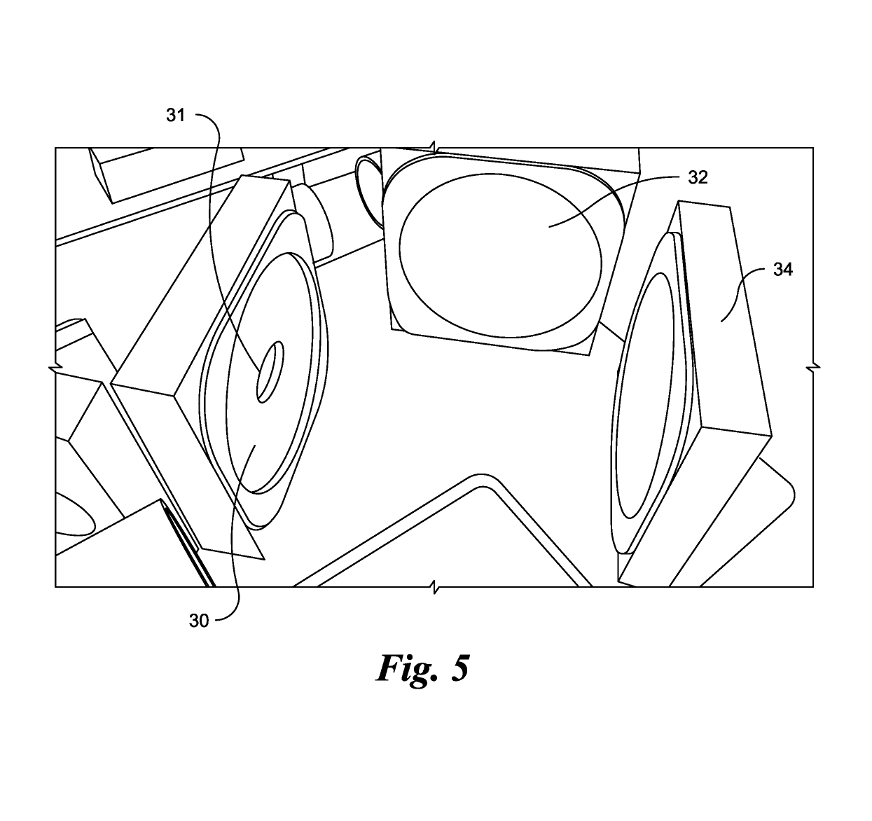

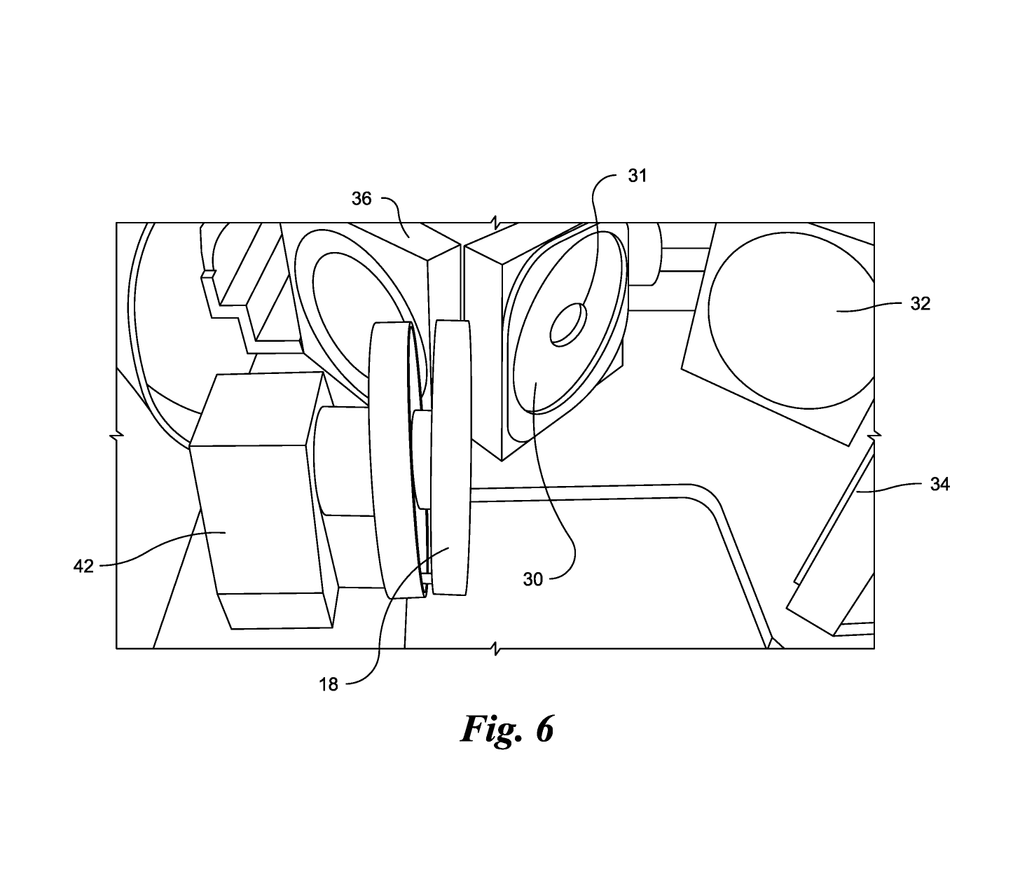

Referring to the drawings in detail, wherein like reference numbers identify like structure throughout, FIGS. 1-7 show a device, generally designated 10, in accordance with one embodiment the present disclosure. The device 10 can be configured to direct light toward or to a subject (e.g., a patient), receive light reflected or refracted from the subject, and analyze the received light to determine if the subject has experienced a brain injury. To accomplish such functionality, the device 10 can include at least one, two or four or more spaced-apart lights 12 on an exterior surface of a body (e.g., a front face) thereof (see FIGS. 1 and 2). The body of the device can include a first or top side or surface, an opposing second or bottom side or surface, the front or first face, and an opposing rear or second face. When in use, the front face can be directed toward the subject that may have suffered an injury, and the rear face can be directed toward the individual holding the device.

The lights 12 can be in the form of light-emitting diodes (“LEDs”) designed to capture the subjects attention when lit, for example. Each light 12 can illuminate to display a different color (e.g., yellow, orange, red and green). The lights 12 can be illuminated at the same or at different times (e.g., in sequence or in a manner that appears random to the subject). For example, the lights 12 can illuminate in the order shown by numbers 1, 2, 3 and 4 in FIG. 2. Thus, in operation, the lights 12 can function as moving fixation targets that the subject or patient is required or requested to acquire or follow with their eye(s) to complete a specific task or routine. To protect against test subjects or patients anticipating the target sequence and manipulating results, the saccadic trigger can be varied based upon at least two variables: 1) the location of the illuminated light (L₀, . . . , L₄), and/or 2) the time interval between light illuminations (t₀, . . . ,t₀₊ₙ). The subject could simply follow the illumination pattern, or one or more other signals (e.g., audible signal(s)) could be given (e.g., from the device 10) to instruct or help the subject follow the pattern.

The lights 12 can surround or be positioned proximate to a target 14. In one embodiment, the target 14 can include an image of a smiley face, or can be a light that illuminates in the form of a smiley face. However, the target 14 is not limited to the exact size or form shown and described herein. The target 14 can be visible from outside the device 10 through an opening or window 15 in the device 10. The opening 15 can have a diameter that is exactly the same as or at least slightly larger than a diameter of each of the lights 12. In one embodiment, illumination of the lights 12 and the target 14 can be synchronized to measure saccadic latency of the patient.

The device 10 can be sized, shaped, and/or configured to be portable and easily transported and stored. For example, the device 10 can be hand-held or sufficiently small for an individual to pick-up the device with his or her hands. The device 10 can be used by pediatricians, coaches, medics and the like. For example, the device 10 can be helpful immediately after a car accident, on a playing field shortly after a head-related injury, or by a soldier on the battlefield after a fellow soldier is wounded. In one embodiment, after immediate needs of the subject are satisfied (e.g., making sure that there has been no injury to the subject's spinal cord), the device 10 can be used to determine if the patient has suffered a head or brain injury. For example, a first individual can hold the device 10 so that a second, possibly injured individual can view the lights 12 and the target 14. During operation, in one embodiment, the distance between the device 10 and the second, possibly injured individual can be sufficiently small, such as approximately 400 mm or 1 foot, so that the user's eyes must move an appreciable distance when following the lights 12 and/or the target 14. The first individual can hold the device 10 by one or more handles 40 on the exterior thereof (e.g., handles on opposing sides of the device 10). Each handle 40 can extend outwardly from a side of the device 10. The second individual can be instructed by the first individual to watch the lights 12 and/or the target 14 and follow the lights 12 and/or the target 14 as each are illuminated, for example.

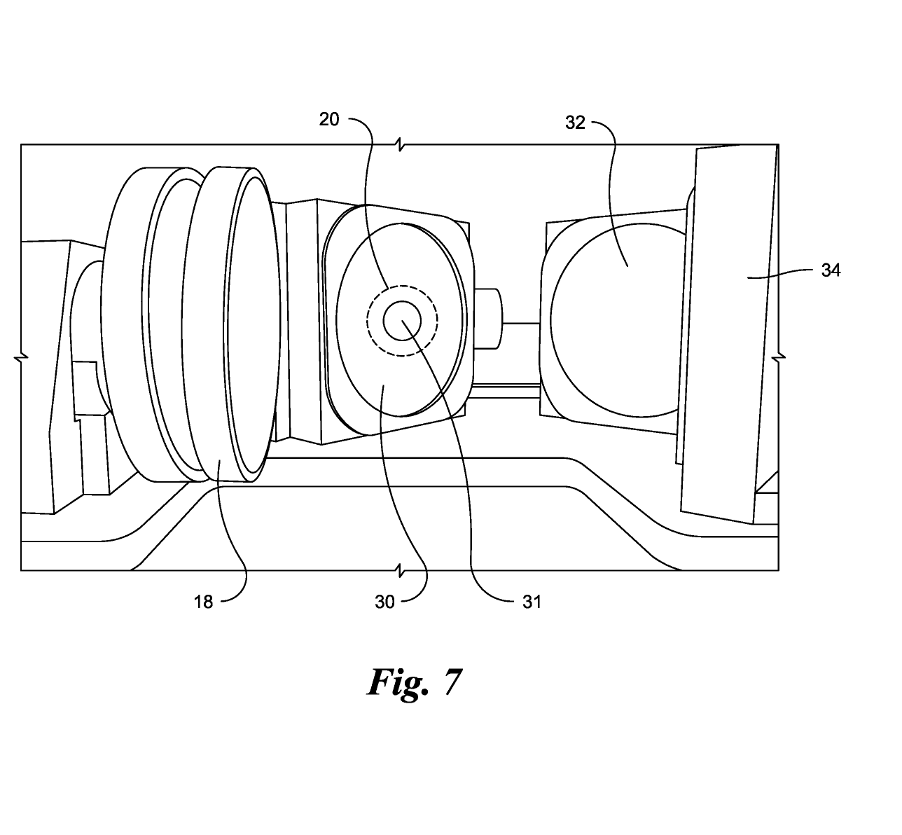

In one embodiment of the present disclosure, the birefringence scanning method described in U.S. Pat. No. 6,027,216 can be optimized or improved by the device 10. In one embodiment, within the device 10, a light source 28 (e.g., a laser) can produce a beam of low-power (e.g., less than 50 mW), diverging, polarized laser light. At least some of the light produced by the light source 28 can be directed or reflected onto a tilted, spinning mirror 18 (i.e., “the first mirror”) to create a scan 20 on a second mirror 30 (see FIG. 7). Use of “first,” “second,” “third,” etc. herein when referring to the mirrors does not have any relation to the order in which light from the light source 28 or from the possibly injured individual travels reflects off of or contacts the mirrors. The scan 20 can appear to the subject to be in the shape of a circle, which can be the result of the straight or “spot” light beam from the light source 28 contacting the spinning first mirror 18. In other words, the scan 20 can give the illusion of a ring because the spot from the light source 28 is swept out at a sufficient rate that it appears to the user to be a ring. The scan 20 can be directed toward one or both eyes of the subject, and the scan 20 and the smiley face of the target 14 can be visible by the subject at the same time. If the smiley face of the target 14 is being observed properly by the subject, the scan 20 sweeps out and hits fibers in the eye. In one embodiment, polarization can change twice for every one light sweep.

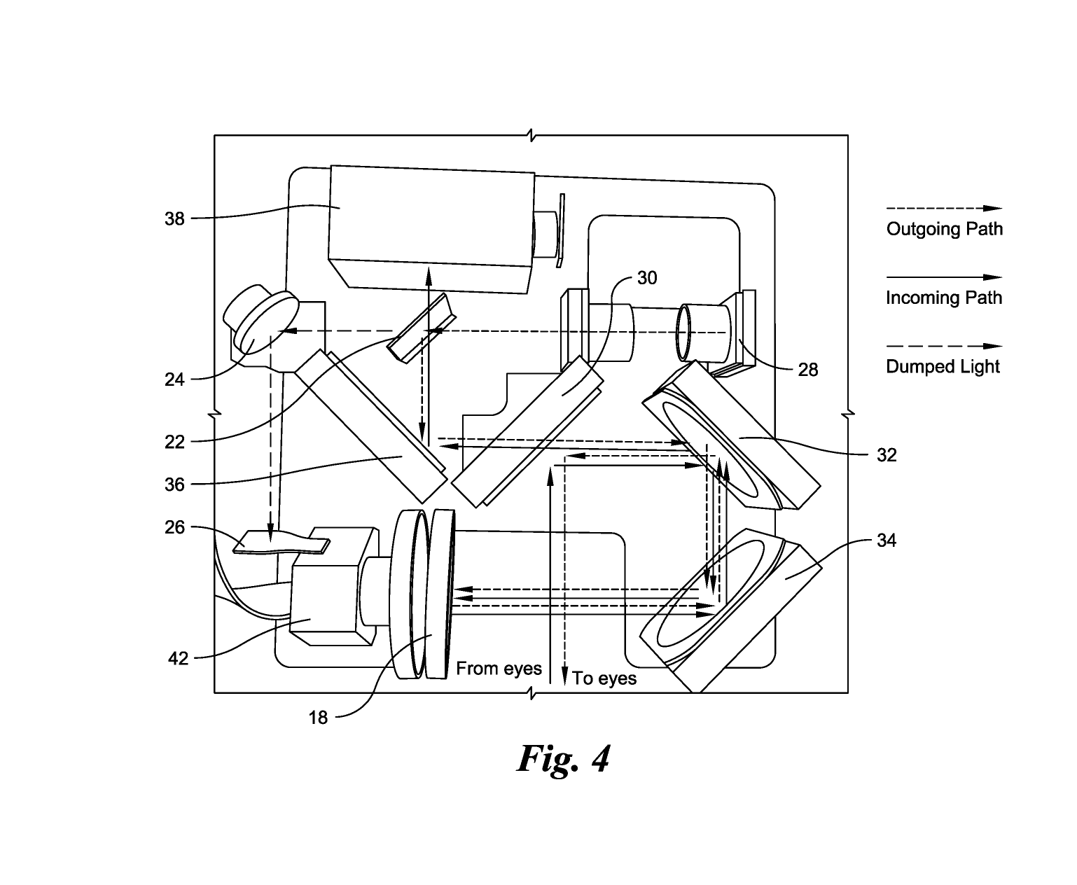

The first mirror 18 can have a balanced mass design for lower cost. The first mirror 18 can have an at least slightly concave front face. The first mirror 18 can be at least slightly tilted with respect to a plane in which the light from the light source 28 travels. The first mirror 18 can be operatively attached to a motor 42, which is configured to selectively rotate or spin the first mirror 18. The second mirror 30 can include a toroidal curvature. More particularly, the second mirror 30 can have a concave shape, with a radius of curvature different in the horizontal axis as compared with the vertical axis (both curvatures being concave). The second mirror 30 can include a first aperture or hole 31, such as at a center thereof. The first aperture 31 can be in the shape of a circle. The smiley face of the target 14 can be aligned with or visible by the subject on or inside the first aperture 31 when the device 10 is positioned appropriately with respect to the subject. In one embodiment, the first aperture 31 can be a glass surface on which the target 14 can be located. The second mirror 30 can include a second aperture or hole, such as at a center thereof. The second aperture can be generally perpendicular to the first aperture, but can share at least some common space. The second aperture allows at least a portion of the beam of light to travel therethrough (see FIG. 4).

The subject can fixate on a target (e.g., target 14) within or on the device 10, which can center the circle on the point of retinal fixation and can focus the laser light onto the retina during the scan. The light emanating from the device 10 can pass through the nerve fibers of the retina of the subject, and light can be altered by the retinal structure and ocular alignment at the moment of the scan. The incident light can then be retro-reflected by the fundus of the eye and returned through the optical system of the eye back to the device 10.

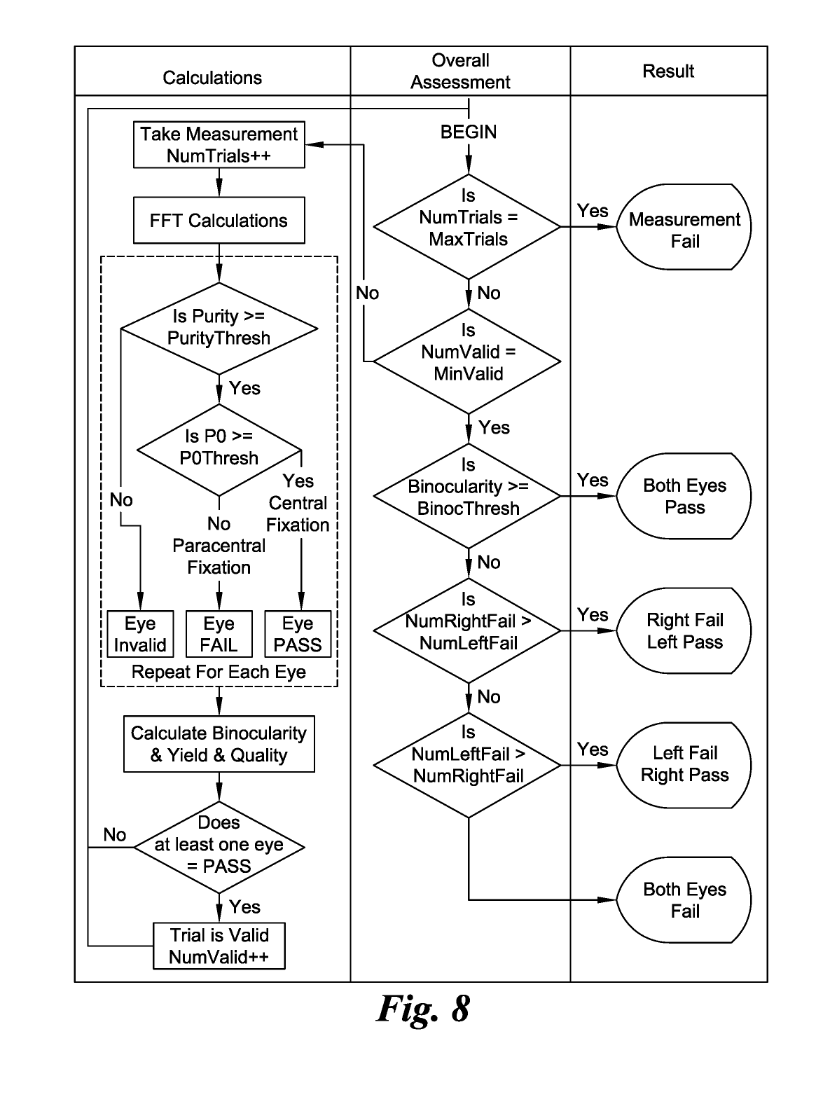

In one embodiment, the returning light can enter through the opening 15 of the device 10, can be reflected within the device 10 and directed a haplopscopic knife-edge prism or set of mirrors within a detector block 38 to separate the right and left eye signals. Each beam path can then be directed onto right and left eye polarization analyzers or one or more polarization sensitive detectors within the detector block 38. Changes in polarization can be converted to an electrical signal and digitized for real-time analysis using onboard software and proprietary algorithms. One example of such an algorithm is shown in FIG. 8. In one embodiment, the prism or set of mirrors can be two mirrors positioned at 90 degrees with respect to each other so as to direct light in two different directions.

FIG. 4 shows the path of light according to one embodiment of the present disclosure. For example, light emanating from the light source 28 can be directed to a beamsplitter 22. The light may travel between or through two or more optical components between the light source 28 and the beamsplitter 22. In one embodiment, the beamsplitter 22, or power splitter, is an optical device configured to split an incident light beam (e.g., a laser beam) into two or more beams, which may or may not have the same optical power. The beamsplitter 22 can appear to be transparent or translucent. The beamsplitter 22 can define a plane that extends at an angle (e.g., approximately 45 degrees) from the direction in which the light source 28 sends the path or beam of light. Light reflected from the beam splitter 22 can contact a third mirror 36. Light reflected from the third mirror 36 can contact a fourth mirror 32, such that the light can travel through the second aperture of the second mirror 30. Light reflected from the fourth mirror 32 can contact a fifth mirror 34. Light reflected from the fifth mirror 34 can contact the spinning first mirror 18. Light reflected from the first mirror can be directed back to the fifth mirror 34, then to the fourth mirror 32, then to the second mirror 30, and out the opening 15 of the device 10 and to the subject. In one embodiment, some of the mirrors of the presently disclosed technology, such as the third mirror 36, the fourth mirror 32 and the fifth mirror 34 can be either dielectric mirrors or gold mirrors. Sometimes, dielectric mirrors can generate high stray light due to scatter, so it can be desirable in those circumstances to use gold mirrors. The gold mirrors can include a surface of reflection made of gold. The bulk of the material used to make a gold mirror can be glass, with a precision ground and polished flat surface. The flat surface can have a thin coating of metals applied, with gold as the final metal. The reflection properties of a gold mirror are dictated by the gold coating.

As shown in FIG. 4, light returning to the device 10 from the subject can generally follow the reverse path as light generated from the device 10 or the light source 28. However, light reflected from the third mirror 36 flows through the beamsplitter 22 and into the detector block 38, which can include polarization sensitive detectors therein.

In one embodiment, a software analysis can begin with a computerized Fourier analysis of the digitized signal to identify frequency components. If the frequency of returning light for any one eye is determined to have doubled during its passage through the eye, central fixation of the fovea of that eye is confirmed. If central fixation is detected in both eyes simultaneously, the subject is said to have normal binocular alignment and normal binocular vision. If central fixation is not detected in one or both eyes, then the subject is determined to have a disruption in the alignment.

As compared to the device disclosed in U.S. Pat. No. 6,027,216, the presently disclosed technology can include one or more of modifications, such as inter-pupillary distance (“IPD”), illumination levels, focus sensitivity, saccadic latency, and software improvements.

IPD: Prior art Pediatric Vision Scanners (“PVS”) can be optimized for an IPD of 50 mm. However, the average young adult has an IPD of 63 mm. Increasing the IPD requires repositioning of the differential polarization sensors and apertures within the instrument, such that both exit pupils are 40 mm wide and separated by 23 mm between the nasal edges. This optomechanical alteration permits the instrument of the present disclosure to capture more light from the eyes, enhancing signal to noise ratio and improving diagnostic capability, without removing the capability of testing on children (minimum IPD 40 mm at age 5). In one embodiment of the present disclosure, the device 10 can be modified to accommodate an IPD for both adults and children (e.g., two positions) in a single device. In another embodiment of the present disclosure, one device 10 can be sized for the IPD of adults, and a second device 10 can be sized for the IPD of children.

Illumination levels: As individuals age, the reflectivity of their eye tends to decline gradually due to subclinical lens opacification and reduction in cone photoreceptor nerve fiber layer thickness. This decline reduces the amount of reflected light available for the device. Prior art PVS use extremely low light levels (e.g., 240 μW), allowing for infinite exposure even with a stationary laser. There is ample room within the current safety standards to increase the intensity of illumination and thus improve signal strength of returning light. One way to achieve this goal is to modify the beamsplitter. In prior art devices, the main beamsplitter (a 50:50 type) dumps half the energy from the laser, as well as half the energy returning from the eyes—a necessary loss of light because the outgoing and returning light paths must be coaxial in order to perform the measurements. In one embodiment, the device of the present disclosure provides an improvement to the returning light path by moving toward a brighter source laser, while remaining within International Electrotechnical Commission (“IEC”) laser safety limits, and dumping a larger portion of the modified laser energy. In the device of the present disclosure, utilizing a 90:10 beamsplitter 22 can dump 90% of the laser energy (e.g., light created by the light source 28), but permits 90% of the energy from the eyes (e.g., light returned from the subject's eyes) to return to the sensors within the detector block 38. Stated differently, the bean dump design of the presently disclosed technology passes or suppresses approximately 90% of the energy from the light source 28 while reflecting approximately 10%. The beam dump design of one embodiment of the presently disclosed technology includes a first component 24 and a second component 26 spaced-apart therefrom. The first and second components 24, 26 receive, dispose of and/or absorb light from the light source 28 that travels through the beamsplitter 22. Enablement of this design improvement requires improvements to the beam dumps to ensure that the light does not back scatter along the return path. Such a design also allows for the use of a more powerful (e.g., brighter) light source 28, without the subject noticing the additional light.

Focus sensitivity: The original pediatric device performance was enhanced by detection of poor focus of returning light. For the device of the present disclosure, a goal is to noninvasively assess fixation stability and saccade velocity even when the eyes are out of focus. Specifically, the device of the present disclosure can enable accurate evaluation of index metrics in individuals with uncorrected refractive error. To reduce the sensitivity of the instrument to defocus, the lens assembly and at least one aperture can be altered to permit a larger returning beam. Increasing the size of the lenses is likely to increase background signal levels. The optical system of the present disclosure can include additional features or measures to control background signals.

In prior art devices, the largest sources of background signals are generated within the instrument. The device and/or method of the present disclosure reduce the background signals by migrating to a more rigorous overall stray-light control scheme. According to the device and/or method of the present disclosure, prophetic methods to reduce the stray light background signal levels include: (1) use of a position-encoded motor to enhance synchronization of the background signal with the data signal, and/or (2) precise positioning of apertures, and (3) reconfiguration of beam dumps. In one embodiment, the beams dumps can be moved as far away as possible from the detector block, in relation to the apertures. The device and/or method of the present disclosure can reduce the background signal levels by at least a factor of 10, thereby allowing a significant increase in the size of the beam returning from the eye.

Saccadic latency: Prior art Retinal Birefringence Scanning (“RBS”) devices do not incorporate saccadic latency measures. One embodiment of the present disclosure includes surrounding the existing scanning beam with a plurality, such as four (4) to six (6), of evenly spaced-apart LEDs 12 at a periphery of a viewing area (see, e.g., FIG. 1). Each LED can project or display a different color (e.g., red, green, yellow, orange). FIG. 2 includes numbers to identify each LED in one embodiment of the device 10. Those skilled in the art will understand that such numbers are included herein for convenience and to facilitate description, but are not included on the actual device.

To test latency, the subject or patient could receive an audible and visual cue, look at the illuminated peripheral LEDs (singly or in sequence), and then immediately return gaze to the center fixation target 14. The latency between issuance of the initial peripheral cue and the detection of central fixation could then be recorded. Saccadic latency is expected to be between 250-400 ms depending on the task, so that the full “round trip” latency would be a minimum of 500 ms. The prior art pediatric RBS device has a scanning frequency of 100 Hz, such that central fixation can be detected within 10 scans, or about 100 ms. To further enhance the temporal resolution of fixation detection, motor speed in the device of the present disclosure could be increased accordingly through basic engineering methods.

Software modification: Prior art software allows a maximum of five (5) seconds of testing and does not support the full spectrum of monocular, binocular, fixation stability, binocularity, and saccadic latency testing. To optimize for more comprehensive assessment of fixation stability and saccadic latency, the software of the present disclosure allows for an extended signal capture under monocular or binocular conditions. Software of one embodiment of the present disclosure can step through a series of tests to prompt each of the following:
Binocular fixation stability (10 sec); and
Saccadic latency task (15 sec).

Data storage and testing time will take duration of testing and temporal resolution of the fast Fourier transform (“FFT”) into account, as shown in FIG. 7.

Other modifications: A modification that relates to the above alterations is the creation or use of the second mirror 30 to improve optical power. The second mirror 30 includes a curvature to the surface to create a concave mirror and can eliminate the major existing source of stray light in the device of U.S. Pat. No. 6,027,216. In prior art systems, a refractive lens—even when coated to reduce reflections—will generate reflections that can migrate back to the receiver. In the device of the present disclosure, which can include the toric second mirror 30, a fold mirror and a lens of the prior art are not necessary. There are no refractive surfaces common to outgoing and returning light, so only the desired light from the eyes returns to the sensor receivers. There are no stray light reflections to control. This modification will markedly improve system performance and is accomplished through component replacements.

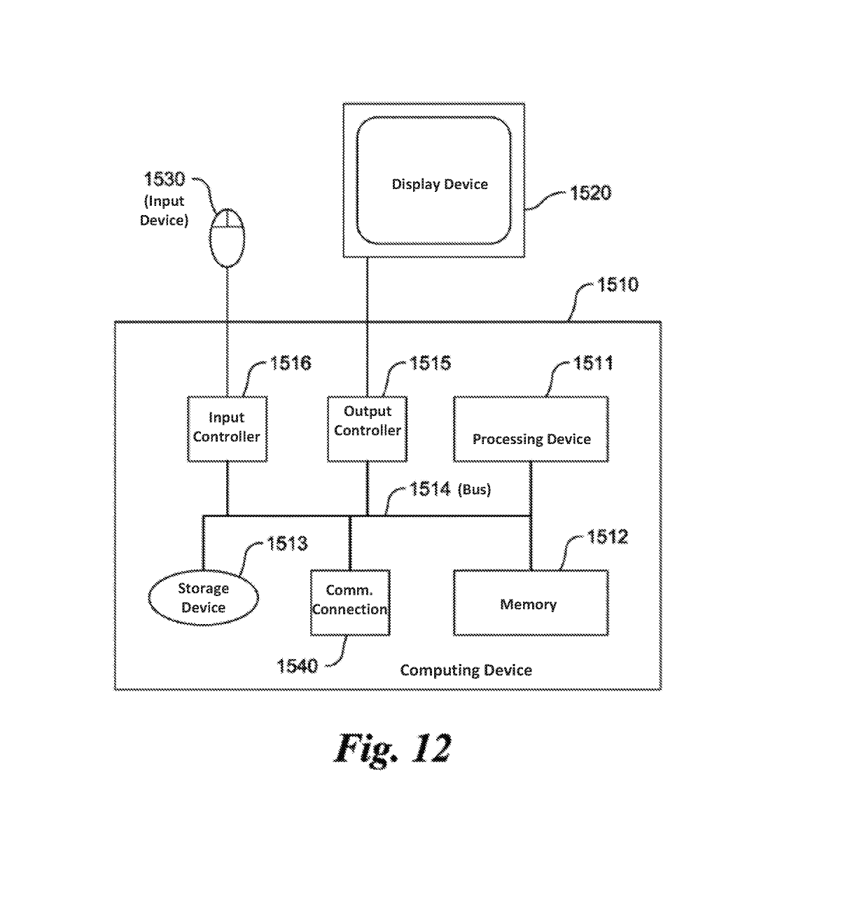

One or more of the above-described systems and/or methods may be implemented with or involve software, for example modules executed on or more computing devices 1510 (see FIG. 12). Of course, modules described herein illustrate various functionalities and do not limit the structure or functionality of any embodiments. Rather, the functionality of various modules may be divided differently and performed by more or fewer modules according to various design considerations.

Each computing device 1510 may include one or more processing devices 1511 designed to process instructions, for example computer readable instructions (i.e., code), stored in a non-transient manner on one or more storage devices 1513. By processing instructions, the processing device(s) 1511 can perform one or more of the steps and/or functions disclosed herein. Each processing device can be real or virtual. In a multi-processing system, multiple processing units can execute computer-executable instructions to increase processing power. The storage device(s) 1513 can be any type of non-transitory storage device (e.g., an optical storage device, a magnetic storage device, a solid state storage device, etc. The storage device(s) 1513 can be removable or non-removable, and include magnetic disks, magnetic tapes or cassettes, CD-ROMs, CD-RWs, DVDs, or any other medium which can be used to store information. Alternatively, instructions can be stored in one or more remote storage devices, for example storage devices accessed over a network or the internet.

Each computing device 1510 additionally can have memory 1512, one or more input controllers 1516, one or more output controllers 1515, and/or one or more communication connections 1540. The memory 1512 can be volatile memory (e.g., registers, cache, RAM, etc.), non-volatile memory (e.g., ROM, EEPROM, flash memory, etc.), or some combination thereof. In at least one embodiment, the memory 1512 can store software implementing described techniques.

An interconnection mechanism 1514, such as a bus, controller or network, can operatively couple components of the computing device 1510, including the processor(s) 1511, the memory 1512, the storage device(s) 1513, the input controller(s) 1516, the output controller(s) 1515, the communication connection(s) 1540, and any other devices (e.g., network controllers, sound controllers, etc.). The output controller(s) 1515 can be operatively coupled (e.g., via a wired or wireless connection) to one or more output devices 1520 (e.g., a monitor, a television, a mobile device screen, a touch-display, a printer, a speaker, etc.) in such a fashion that the output controller(s) 1515 can transform the display on the display device 1520 (e.g., in response to modules executed). The input controller(s) 1516 can be operatively coupled (e.g., via a wired or wireless connection) to an input device 1530 (e.g., a mouse, a keyboard, a touch-pad, a scroll-ball, a touch-display, a pen, a game controller, a voice input device, a scanning device, a digital camera, etc.) in such a fashion that input can be received from a user.

The communication connection(s) 1540 enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

FIG. 12 illustrates the computing device 1510, the output device 1520, and the input device 1530 as separate devices for ease of identification only. However, the computing device 1510, the display device(s) 1520, and/or the input device(s) 1530 can be separate devices (e.g., a personal computer connected by wires to a monitor and mouse), can be integrated in a single device (e.g., a mobile device with a touch-display, such as a smartphone or a tablet), or any combination of devices (e.g., a computing device operatively coupled to a touch-screen display device, a plurality of computing devices attached to a single display device and input device, etc.). The computing device 1510 can be one or more servers, for example a farm of networked servers, a clustered server environment, or a cloud services running on remote computing devices.

It will be appreciated by those skilled in the art that changes could be made to the embodiments described above without departing from the broad inventive concept thereof. For example, the steps or order of operation of one of the above-described methods could be rearranged or occur in a different series, as understood by those skilled in the art. It is understood, therefore, that this disclosure is not limited to the particular embodiments disclosed, but it is intended to cover modifications within the spirit and scope of the present disclosure as defined by the appended claims.

We claim:

1. A device for measuring saccadic latency, the device comprising:
a light source positioned within a housing and configured to project a beam of light through a window of the housing and onto at least one retina of a subject located external to the housing;
one or more lights disposed on or in an external surface of the housing;
a controller configured to cause output of a sequence of one or more cues, each cue in the sequence of cues corresponding to either a light in the one or more lights or a fixation target that is configured to appear co-axial with the beam of light to the subject located external to the housing;
one or more detectors positioned within the housing and configured to detect light reflected from the at least one retina of the subject through the window of the housing;
wherein the controller is further configured to determine at least one time interval corresponding to at least one delay between output of at least one cue in the sequence of cues and either fixation or loss of fixation by the subject and wherein the fixation or loss of fixation is determined based at least in part on the light reflected from the at least one retina of the subject.

2. The device of claim 1, further comprising:
a first mirror positioned within the housing and configured to reflect light received from the light source, at least a portion of the first mirror being configured to rotate and thereby create a scan on a second mirror positioned within the housing, the second mirror being configured to receive light reflected from the first mirror and reflect the received light through the window of the housing.

3. The device of claim 2, wherein the first mirror comprises a concave face and wherein the second mirror comprises a toroidal curvature.

4. The device of claim 3, wherein a radius of curvature of the second mirror in a horizontal axis is different than a radius of curvature of the second mirror in a vertical axis.

5. The device of claim 2, wherein the fixation target is aligned with an aperture in the second mirror along a beam path of the beam of light.

6. The device of claim 1, wherein the one or more lights comprise a plurality of lights.

7. The device of claim 1, wherein the at least one of the one or more lights is a light-emitting diode.

8. The device of claim 1, wherein the sequence of cues comprises one or more of: an audio cue or a visual cue.

9. The device of claim 8, wherein the audio cue comprises an audible instruction to fixate on either the fixation target or one of the one or more lights.

10. The device of claim 8, wherein the visual cue comprises either activation or deactivation of either the fixation target or one of the one or more lights.

11. The device of claim 1, wherein the beam of light comprises a polarized beam of light and wherein the one or more detectors comprise one or more polarization sensitive detectors.

12. The device of claim 1, wherein the controller is further configured to determine either fixation or loss of fixation by the subject based at least in part on a comparison of the beam of light with the light reflected from the at least one retina of the subject.

13. A method for measuring saccadic latency, the method comprising:
projecting, by a light source positioned within a housing, a beam of light through a window of the housing and onto at least one retina of a subject located external to the housing;
outputting, by a controller, a sequence of one or more cues, each cue in the sequence of cues corresponding to either a light in one or more lights disposed on or in an external surface of the housing or a fixation target that is configured to appear co-axial with the beam of light to the subject located external to the housing;
detecting, by one or more detectors positioned within the housing, light reflected from the at least one retina of the subject through window of the housing; and
determining, by the controller, at least one time interval corresponding to at least one delay between output of at least one cue in the sequence of cues and either fixation or loss of fixation by the subject, wherein the fixation or loss of fixation is determined based at least in part on the light reflected from the at least one retina of the subject.

14. The method of claim 13, further comprising:
reflecting, by a first mirror positioned within the housing, light received from the light source, at least a portion of the first mirror being configured to rotate and thereby create a scan on a second mirror positioned within the housing, the second mirror being configured to receive light reflected from the first mirror and reflect the received light through the window of the housing.

15. The method of claim 14, wherein the first mirror comprises a concave face and wherein the second mirror comprises a toroidal curvature.

16. The method of claim 15, wherein a radius of curvature of the second mirror in a horizontal axis is different than a radius of curvature of the second mirror in a vertical axis.

17. The method of claim 14, wherein the fixation target is aligned with an aperture in the second mirror along a beam path of the beam of light.

18. The method of claim 13, wherein the one or more lights comprise a plurality of lights.

19. The method of claim 13, wherein the at least one of the one or more lights is a light-emitting diode.

20. The method of claim 13, wherein the sequence of cues comprises one or more of: an audio cue or a visual cue.

21. The method of claim 20, wherein the audio cue comprises an audible instruction to fixate on either the fixation target or one of the one or more lights.

22. The method of claim 20, wherein the visual cue comprises either activation or deactivation of either the fixation target or one of the one or more lights.

21. The method of claim 13, wherein the beam of light comprises a polarized beam of light and wherein the one or more detectors comprise one or more polarization sensitive detectors.

22. The method of claim 13, wherein the controller is further configured to determine either fixation or loss of fixation by the subject based at least in part on a comparison of the beam of light with the light reflected from the at least one retina of the subject.

23. A device for neurological screening for assessing brain dysfunction of a subject, the device comprising:
a housing having a window therein;
a light source positioned within the housing and configured to produce a beam of light, at least a portion of the beam of light being configured to pass through the window and onto at least one retina of at least one eye of the subject;
one or more photodetectors within the housing configured to detect one or more reflections of the beam of light from the at least one retina through the window;
one or more lights positioned on an exterior of the housing;
a target configured to appear on the exterior of the housing at the window; and
a controller configured to illuminate the one or more lights in a known sequence, assess the one or more reflections of the beam of light from the at least one retina to determine eye fixation of the at least one eye of the subject, and determine a time to achieve fixation or loss of fixation of the at least one eye in relation to the target and a change in illumination of the one or more lights.

24. The device of claim 23, wherein the controller is further configured to:
compare the time to achieve fixation or loss of fixation with an expected time; and
determine whether the subject passes or fails a test for assessing a brain dysfunction based at least in part on the comparison.

25. The device of claim 24, wherein the brain dysfunction comprises traumatic brain injury.

26. A neurological screening method for assessing brain dysfunction of a subject, the method comprising:
positioning a window of a housing of a neurological screening device in front of at least one eye of the subject;
producing, by a light source positioned within the housing, a beam of light, at least a portion of the beam of light being configured to pass through the window and onto at least one retina of the subject;
detecting, by one or more photodetectors within the housing, one or more reflections of the beam of light from the at least one retina through the window;
illuminating, by a controller, one or more lights positioned on an exterior of the housing in a known sequence;
assessing, by the controller, the one or more reflections of the beam of light from the at least one retina to determine eye fixation of the at least one eye of the subject; and
determining, by the controller, a time to achieve fixation or loss of fixation of the at least one eye in relation to a target configured to appear on the exterior of the housing at the window and a change in illumination of the one or more lights.

27. The neurological screening method of claim 26, further comprising:
comparing, by the controller, the time to achieve fixation or loss of fixation with an expected time; and
determining, by the controller, whether the subject passes or fails a test for assessing a brain dysfunction based at least in part on the comparison.

28. The neurological screening method of claim 27, wherein the brain dysfunction comprises traumatic brain injury.
